(19) United States
(12) Patent Application Publication
(10) Pub. No.: US 2011/0217234 A1
(43) Pub. Date: Sep. 8, 2011

(54) IMAGING LIGANDS

(76) Inventors: Ravi Hegde, Bangalore (IN); Chandan Atreya, Bangalore (IN); Bo Shan, Shanghai (CN); Sudeshna Adak, Bangalore (IN)

(21) Appl. No.: 13/128,418

(22) PCT Filed: Nov. 20, 2009
(86) PCT No.: PCT/US09/65268

§ 371 Date: May 10, 2011

Related U.S. Application Data
(62) Division of application No. 61/117,254, filed on Nov. 24, 2008.

(30) Foreign Application Priority Data
Nov. 24, 2008 (GB) 0821432.2

(51) Int. Cl. A61K 51/04; C07D 265/34

(52) U.S. Cl. 424/1.89; 544/101

(57) ABSTRACT

Naphthoxazine derivatives which are selective ligands for the dopamine D2 receptor and which carry an 18F radio-label suitable for imaging with PET are described. The compounds of the present invention are thus useful for in vivo diagnostics and in vivo imaging of the dopamine D2 receptor.

The present invention relates to the field of medical diagnostics and imaging using positron emission tomography (PET) and provides compounds and methods for visualising central nervous system (CNS) receptors. In particular, this invention relates to naphthoxazine derivatives which are selective ligands for the dopamine D2 receptor and which carry an ¹⁸F-radiolabel suitable for imaging with PET. The compounds of the present invention are thus useful for in vivo diagnostics and in vivo imaging of the dopamine D2 receptor.

Dopamine is an important neurotransmitter in the human brain. Dysfunctions of dopamine neurotransmission are implicated in many neurological and psychiatric disorders - for example, schizophrenia, Parkinson's Disease, psychosis, anxiety, and Attention Deficit Hyperactivity Disorder (ADHD). In addition to its role in the CNS, dopamine agonists may also be used to increase cardiac output and blood pressure in patients with shock and heart failure. Current evidence indicates that changes in the receptors which mediate dopamine transmission are associated with particular CNS (central nervous system) disorders. Dopamine receptors fall into two main types : D1-like and D2-like receptors, within which there are several receptor sub-types. Study of the D2 dopamine receptor is considered particularly valuable for assistance in diagnosis and therapy monitoring of the neurological disorders mentioned, as well as for clinical research on healthy human volunteers and for therapeutic drug development trials.

A number of compounds have been investigated as potential radioligands for studying the dopamine D2 receptor in vivo using PET including [¹¹C]-4-propyl-2H-naphth[1,2-b][1,4]oxazin-9-ol ([¹¹C]-PHNO), [¹¹C]-N-methylspiperone, [¹¹C]-raclopride, [¹²³I]-iodobenzamide, [¹²³I]-epidipride, [¹⁸F]-fallypride, and [¹¹C]-FLB-457.

Certain ¹⁸F-labelled derivatives of PHNO have been described in WO2006/084368 having the structure:

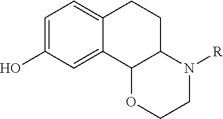

wherein R is C₁₋₆alkyl in which one hydrogen atom on the alkyl chain is replaced with fluoro or radioactive fluoro. But these compounds have performed poorly in vivo and ex vivo.

In in vivo biodistribution studies in rats, [¹⁸]F-PHNO showed rapid uptake to the brain, but no regional specificity between D2-rich areas of the brain (e.g. striata) and areas of low D2 receptor expression. Similarly, in ex vivo autoradiography studies, [¹⁸]F-PHNO binding in the striata of the rat brain was indistinguishable from the background, non-dopaminergic regions. The authors suggested that fast kinetics and lack of specific binding of [¹⁸]HF-PHNO would preclude its use as a cerebral imaging agent for the D2 receptor. (N. Vasdev et al. Nuclear Medicine and Biology 34 (2007) 195-203).

Therefore, there still exists a need for detectably labelled ligands which can actively target the high affinity state of the D2 receptor (D2High). The present invention seeks to provide detectably labelled ligands suitable for studying the dopamine D2 receptor in vivo having improved properties over those in the prior art.

According to the invention, there is provided a compound of formula (I):

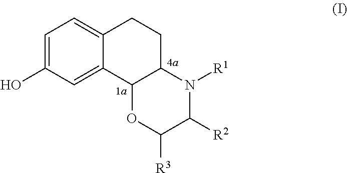

or a salt or solvate thereof; wherein

R¹ is C₁₋₆alkyl; one of R² and R³ is [¹⁸F]fluoro and the other is hydrogen.

In a compound of formula (I), and in following aspects of the invention R¹ is preferably ethyl, n-propyl, or iso-propyl; and is most preferably n-propyl.

Compounds of formula (I) exist in different optical isomer forms, the invention encompasses all such isomers either in substantially pure form, or admixed in any proportion, including racemic mixtures. “Substantially pure form” means that the compound is enantiomerically enriched and comprises at least 95 mole % of a given isomer. In one embodiment, the oxygen and nitrogen of the heterocyclic ring are in a trans configuration. In a further embodiment, both positions 1a and 4a of the compound of formula (I) have an R-configuration.

Thus, in a preferred aspect of the invention, there is provided a compound of formula (I) which is of formula (Ia):

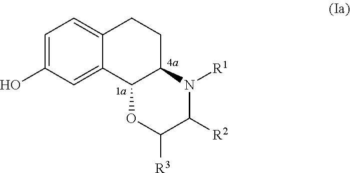

or a salt or solvate thereof, wherein R¹, R², and R³ are as defined above for formula (I).

Preferred specific compounds of formula (I) include:

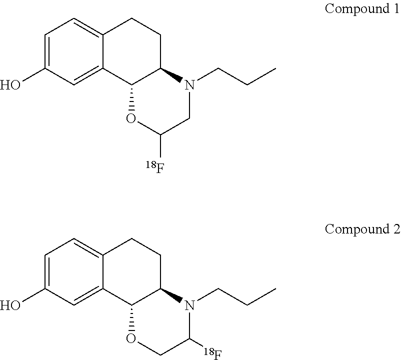

or a salt or solvate thereof.

Said compounds of formula (Ia), Compound 1, and Compound 2 or a salt or solvate thereof, are suitably in substantially pure form.

Suitable salts according to the invention include (i) physiologically acceptable acid addition salts such as those derived from mineral acids, for example hydrochloric, hydrobromic, phosphoric, metaphosphoric, nitric and sulphuric acids, and those derived from organic acids, for example tartaric, trifluoroacetic, citric, malic, lactic, fumaric, benzoic, glycollic, gluconic, succinic, methanesulphonic, and para-toluenesulphonic acids; and (ii) physiologically acceptable base salts such as ammonium salts, alkali metal salts (for example those of sodium and potassium), alkaline earth metal salts (for example those of calcium and magnesium), salts with organic bases such as triethanolamine, N-methyl-D-glucamine, piperidine, pyridine, piperazine, and morpholine, and salts with amino acids such as arginine and lysine.

Suitable solvates according to the invention include those formed with ethanol, water, saline, physiological buffer and glycol.

As used herein the term “alkyl” either alone or as part of another term means a straight, branched or cyclic alkyl group.

As demonstrated below, the compounds of formula (I) have use as PET ligands for the D2 receptor. Therefore, according to a further aspect of the invention, there is provided a compound of formula (I) as defined above, or a salt or solvate thereof, for use in medicine, particularly for use in an in vivo PET diagnostic or imaging method. Suitably, a compound of formula (I) as defined above, or a salt or solvate thereof may also be used to image the D2 receptor in healthy human volunteers for example for clinical research purposes.

According to one aspect of the invention, there is provided a method for detection of D2 receptors in a subject, comprising:
(i) administration of a compound of formula (I) as defined above, or a salt or solvate thereof to said subject; and
(ii) detecting uptake of said compound by in vivo PET imaging.

Such a method provides information and data having utility in the diagnosis and clinical research of D2-mediated disorders. The subject is a mammal, most suitably a human who has or is suspected of having a D2-mediated disorder. The method may be performed quantitatively such that the amount or change in amount of D2 receptors or the density or change in density of receptors in the high-affinity D2High state, may be determined so as to diagnose or track progress of a disease. Alternatively the method may be used to locate D2 receptors.

In a further aspect, there is provided a method for detection of D2 receptors in a subject, comprising:
(i) administration of a compound of formula (I) as defined above, or a salt or solvate thereof to said subject;
(ii) detecting uptake of said compound of formula (I) administered in step (i) by in vivo PET imaging;
(iii) allowing a suitable amount of time to pass such that the compound administered in step (i) has radioactively decayed; then
(iv) administration of an effective amount of either (a) a non-radiolabelled dopamine agonist or dopamine mimetic, or (b) a non-radiolabelled dopamine depletor, and contemporaneous administration of a compound of formula (I) or a salt or solvate thereof as defined in step (i) ;
(v) detecting uptake of said compound of formula (I) administered in step (iv) by in vivo PET imaging.

The time allowed to pass in step (iii) is suitably over 10 hours, more suitably at least 16 hours, and more suitably is around 24 hours such that the PET signal from the compound of formula (I) administered in step (i) is no longer detectable. In an alternative aspect of the invention, there is provided a method for detection of D receptors in a subject, comprising:
(i) administration of a compound of formula (I) as defined above, or a salt or solvate thereof to said subject;
(ii) detecting uptake of said compound of formula (I) administered in step (i) by in vivo PET imaging;
(iii) administration of an effective amount of either (a) a non-radiolabelled dopamine agonist or dopamine mimetic, or (b) a non-radiolabelled dopamine depletor;
(iv) detecting uptake of said compound of formula (I) administered in step (i) by in vivo PET imaging.

The term “dopamine mimetic” means a compound which has a biological activity similar to dopamine or which causes a release of dopamine. The non-radiolabelled dopamine agonist or dopamine mimetic used in the above methods is suitably selected from amphetamine, (+)-PHNO, apomorphine and congeners thereof (for example, N-propyl-norapomorphine) and an aminotetralin (such as dihydroxy-2-dimethyl-aminotetralin). In one aspect, the non-radiolabelled dopamine agonist or dopamine mimetic used in the above methods is amphetamine.

The “non-radiolabelled dopamine depletor” is a compound which temporarily and acutely decreases the availability of dopamine in the subject, for example by inhibiting synthesis or release of endogenous dopamine, and is for example a tyrosine hydroxylase inhibitor such as alpha-methyl-para-tyrosine (AMPT).

“Contemporaneous administration” in the above method means that both compounds are administered to the subject such that they are both biologically active in the subject at the same time. In one aspect of the invention, both compounds are administered substantially at the same time i.e. within 30 minutes of each other, or in a single composition comprising both compounds.

Suitably, the compounds of formula (I) or salt or solvate thereof are useful for in vivo imaging of D2 receptors and thus have utility in the imaging or diagnosis of D2-mediated disorders.

The term “D2-mediated disorders” means neurological and psychiatric disorders such as schizophrenia, Parkinson's disease, psychosis, anxiety, and ADHD. One important D2-mediated disorder is schizophrenia.

Accordingly, there is provided a compound of formula (I) or a salt or solvate thereof for use in the in vivo diagnosis or imaging of a D2-mediated disorder.

In a further aspect, there is provided a method for the in vivo diagnosis or imaging of a D2- mediated disorder in a subject, preferably a human, comprising administration of a compound of formula (I) or a salt or solvate thereof and detecting the uptake of said compound by an in vivo PET imaging technique. The method is especially preferred for the in vivo diagnosis or imaging of schizophrenia, Parkinson's disease, psychosis, anxiety, or ADHD. In a further aspect, there is provided a method for in vivo imaging of a D2- mediated disorder in a subject, preferably a human, to whom of a compound of formula (I) or a salt or solvate thereof has been pre-administered and detecting the uptake of said compound by an in vivo PET imaging technique.

The invention further provides a method of monitoring the effect of treatment of a subject, preferably a human with a drug to combat a D2- mediated disorder, said method comprising administering to said subject a compound of formula (I) or a salt or solvate thereof and detecting the uptake of said compound by an in vivo PET imaging technique such as the methods described above, said administration and detection optionally but preferably being effected repeatedly, e.g. before, during and after treatment with said drug.

A compound of formula (I) or a salt thereof is preferably administered for in vivo use in a pharmaceutical formulation comprising the compound of the invention and a pharmaceutically acceptable excipient. A “pharmaceutical formulation” is defined in the present invention as a formulation comprising compound of formula (I) or a salt or solvate thereof in a form suitable for administration to humans. Administration is preferably carried out by injection of the formulation as an aqueous solution. Such a formulation may optionally contain further ingredients such as buffers; pharmaceutically acceptable solubilisers (e.g. cyclodextrins or surfactants such as Pluronic, Tween or phospholipids); pharmaceutically acceptable stabilisers or antioxidants (such as ascorbic acid, gentisic acid or para-aminobenzoic acid).

The effective in vivo dose of a compound of formula (I), (Ia) or a salt thereof will vary depending on the exact compound to be administered, the weight of the patient, and other variables as would be apparent to a physician skilled in the art. Generally, the dose would lie in the range 0.001 μg/kg to 10 μg/kg, preferably 0.01 μg/kg to 1.0 μg/kg.

Compounds of formula (I) may be prepared by [¹⁸]fluorination of the corresponding compound of formula (II):

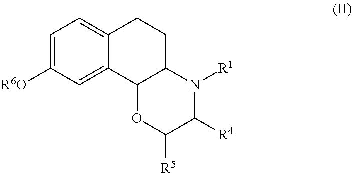

wherein R¹ is as defined for the compound of formula (I) , one of R⁴ and R⁵ is hydrogen and the other is a leaving group (such as a C₁₋₆alkyl-, C₁₋₆haloalkyl- or aryl-sulphonate, suitably methanesulphonate, p-toluenesulphonate, or trifluoromethylsulphonate), and R⁶ is hydrogen or C₁₋₄alkyl preferably methyl.

Compounds of formula (II) are novel and thus form a further aspect of the invention. Preferred compounds of formula (II) include:

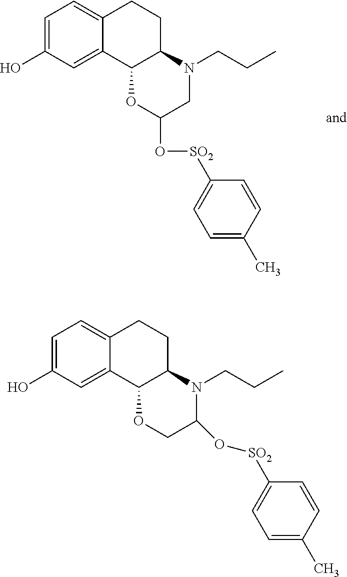

As would be appreciated by a person skilled in the art, protecting groups may be required during synthesis of a compound of formula (I) to prevent unwanted side- reactions. Suitable protecting groups may be found in Protecting Groups in Organic Synthesis, Theodora W. Greene and Peter G. M. Wuts, published by John Wiley & Sons Inc. which describes methods for incorporating and removing such protecting groups.

Fluorination of a compound of formula (II) may be effected by conventional [¹⁸F]radiofluoriation techniques. [¹⁸F]fluoride is conveniently prepared from ¹⁸O -enriched water using the (p,n)-nuclear reaction, (Guillaume et al, Appl. Radiat. Isot. 42 (1991) 749-762) and generally isolated as a salt such as Na¹⁸F, K¹⁸F, Cs¹⁸F, tetraalkylammonium [¹⁸F]fluoride, or tetraalkylphosphonium ¹⁸F fluoride. To increase the reactivity of the [¹⁸F]fluoride, a phase transfer catalyst such as an aminopolyether or crown ether, for example, 4,7,13,16,21,24 hexaoxa-1,10-diazabicyclo[8,8,8] hexacosane (Kryptofix 2.2.2) may be added and the reaction performed in a suitable solvent. These conditions give reactive fluoride ions. Optionally, a free radical trap may be used to improve fluoridation yields, as described in WO 2005/061415. The term “free radical trap” is defined as any agent that interacts with free radicals and inactivates them. A suitable free radical trap for this purpose may be selected from 2,2,6,6-Tetramethylpiperidine-N-Oxide (TEMPO), 1,2-diphenylethylene (DPE), ascorbate, para-amino benzoic acid (PABA), α-tocopherol, hydroquinone, di-t-butyl phenol, β-carotene and gentisic acid.

The treatment with fluoride, suitably [¹⁸F]fluoride may be effected in the presence of a suitable organic solvent such as acetonitrile, dimethylformamide, dimethylsulphoxide, dimethylacetamide, tetrahydrofuran, dioxan, 1,2 dimethoxyethane, sulpholane, N-methylpyrolidininone, or in an ionic liquid such as an imidazolium derivative (for example 1-ethyl-3-methylimidazolium hexafluorophosphate), a pyridinium derivative (for example, 1-butyl-4-methylpyridinium tetrafluoroborate), a phosphonium compound, or tetralkylammonium compound at a non-extreme temperature, for example, 15° C. to 180° C., preferably at elevated temperature, such as 80° C. to 150° C., for example around 120° C.

Representative synthetic pathways for compounds of formula (I) are provided in Schemes 1 and 2 in which the following abbreviations are used: iPr=iso-propyl, n-Pr=n-propyl, Ph=phenyl, TFAA=trifluoroacetic anhydride, DCM=dichloromethane, Ts=tosylate.

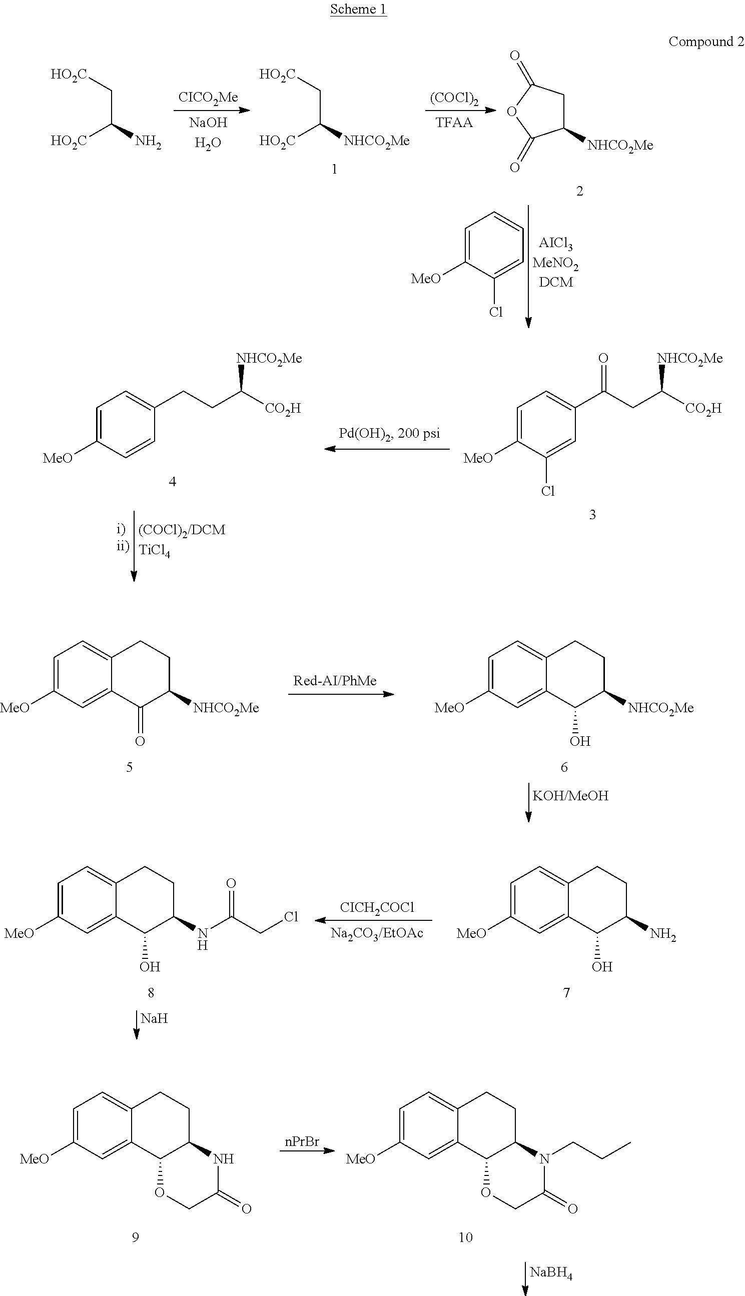

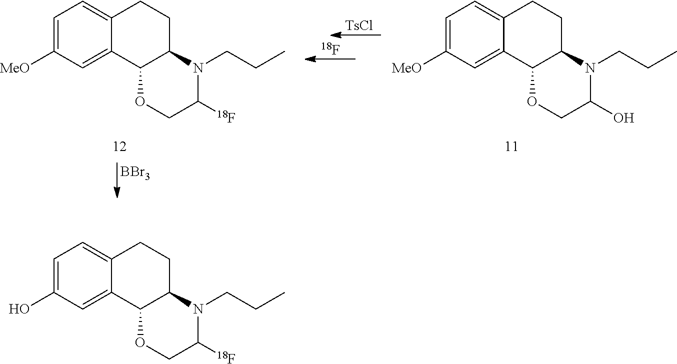

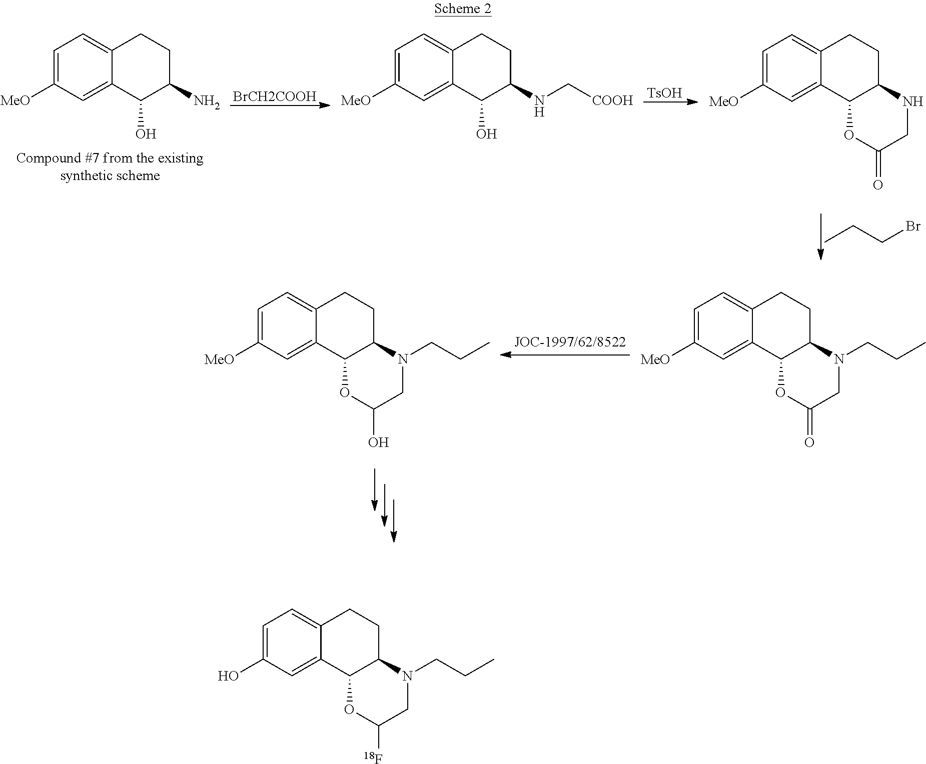

The invention will now be illustrated by way of the Examples in which the following abbreviations are used:
HPLC: high performance liquid chromatography
TLC: thin-layer chromatography
MBq: mega becquerel
Radiofluoridation Example

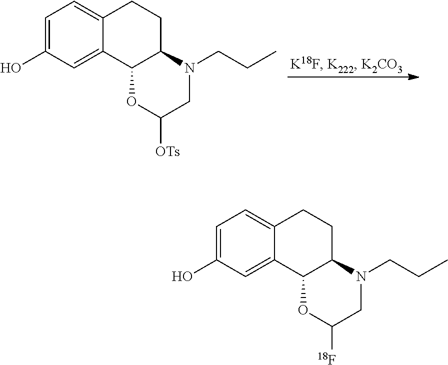

The tosylate precursor is prepared according to Scheme 2 above. The starting materials in scheme 1 are commercially available from Aldrich and the starting material in scheme 2 may be synthesized according to the methods of scheme 1.

[¹⁸F]fluoride (in 200 μL enriched 95% ¹⁸O water), 2.5 mg of Kryptofix 2.2.2 (in 0.5 mL acetonitrile) and 50 μL 0.1M K₂CO₃ are added to a glassy carbon reaction vessel. The solution is then evaporated to dryness using a stream of nitrogen and heating the reaction vessel to 100° C. for 15 minutes. 2×1 mL acetonitrile is added to the reaction vessel at 5 minutes and 10 minutes respectively to aid azeotropic drying. The reaction vessel is cooled to room temperature and the tosylate precursor in 1 mL anhydrous dimethyl sulfoxide is added. The reaction is sealed and heated for 10 minutes at 130° C. The crude mixture is analyzed by HPLC and TLC.

### Biological Examples

To determine in vitro affinities of the compounds, each is tested in an in vitro Human D_{2L} receptor binding assay using Human recombinant (HEK-293) cells and butaclamol as reference compound according to the method of Hall and Strange (1997), British J. Pharmacol., 121: 731-6

Experimental Conditions
Receptor screened: D2L (h)
Origin: Human recombinant (HEK-293 cells)
Competing Ligand: [3H]spiperone (0.3 nM)
Reference control, non-specific binding: butaclamol (10 μM)
Incubation: 60 min/22° C. Scintillation counting

The specific ligand binding to the receptors is defined as the difference between the total binding and the nonspecific binding determined in the presence of an excess of unlabelled ligand. The results are expressed as a percent of control specific binding (measured specific binding/control specific binding)×100) and as a percent inhibition of control specific binding (100−((measured specific binding/control specific binding)×100)) obtained in the presence of the test compounds. The IC50 values (concentration causing a half-maximal inhibition of control specific binding) and Hill coefficients (nH) were determined by non-linear regression analysis of the competition curves generated with mean replicate values using Hill equation curve fitting (Y=D+[(A−D)/(1 +(C/C50)nH)], where Y=specific binding, D=minimum specific binding, A=maximum specific binding, C=compound concentration, C50=IC50, and nH=slope factor). The inhibition constants (Ki) are calculated using the Cheng Prusoff equation (Ki=IC50/(1+(L/KD)), where L=concentration of radioligand in the assay, and KD=affinity of the radioligand for the receptor).

What is claimed is:

1. A compound of formula (I): 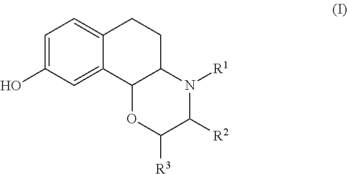
or a salt or solvate thereof; wherein
R¹ is C₁₋₆alkyl;
one of R² and R³ is [¹⁸F]fluoro and the other is hydrogen.

2. A compound according to claim 1 wherein R¹ is ethyl, n-propyl, or iso-propyl.

3. A compound according to claim 1 wherein the oxygen and nitrogen of the heterocyclic ring are in a trans configuration.

4. A compound according to claim 1 which is a compound of formula (Ia): 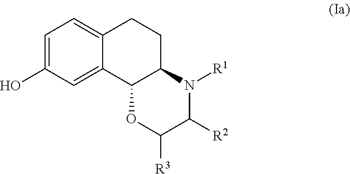
or a salt or solvate thereof, wherein R¹ is C₁₋₆alkyl; and one of R² and R³ is [¹⁸F]fluoro and the other is hydrogen.

5. A compound according to claim 1 which is selected from: 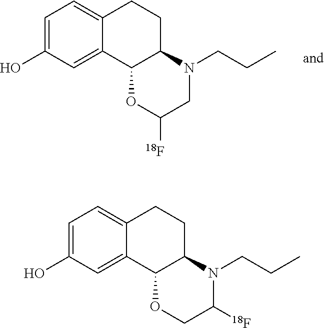
or a salt or solvate thereof.

6. (canceled)

7. (canceled)

8. A method for the in vivo diagnosis or imaging of a D2-mediated disorder in a subject comprising administration of a compound according to claim 1 or a salt or solvate thereof and detecting the uptake of said compound by an in vivo PETimaging technique.

9. A method according to claim 8 wherein the D2- mediated disorder is schizophrenia, Parkinson's disease, psychosis, anxiety, or ADHD.

10. A method for detection of D2 receptors in a subject, comprising:
(i) administration of a compound according to claim 1, or a salt or solvate thereof to said subject; and
(ii) detecting uptake of said compound by in vivo PET imaging.

11. A pharmaceutical formulation comprising a compound according to claim 1 or a salt or solvate thereof, and a pharmaceutically acceptable excipient.

12. A radiolabelling precursor of formula (II) 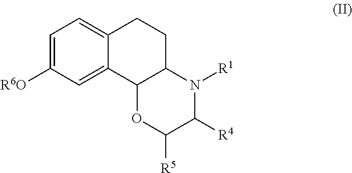
wherein R¹ is as defined in claim 1 and one of R⁴ and R⁵ is hydrogen and the other is a leaving group and R⁶ is hydrogen or C₁₋₄alkyl.

13. The precursor according to claim 12, selected from [chemical structure]
